(19) United States
(12) Patent Application Publication
(10) Pub. No.: US 2003/0138416 A1
(43) Pub. Date: Jul. 24, 2003

(54) Use of glucokinase activator in combination with a glucagon antagonist for treating type 2 diabetes

(76) Inventor: Jesper Lau, Farum (DK)

Correspondence Address: Novo Nordisk Pharmaceuticals, Inc., 100 College Road West, Princeton, NJ, 08540, US

(21) Appl. No.: 10/308,355

(22) Filed: Dec. 3, 2002

Related U.S. Application Data
(60) Provisional application No. 60/336,876, filed on Dec. 5, 2001.
(60) Provisional application No. 60/342,428, filed on Dec. 20, 2001.
(60) Provisional application No. 60/342,355, filed on Dec. 20, 2001.
(60) Provisional application No. 60/386,185, filed on Dec. 21, 2001.
(60) Provisional application No. 60/394,145, filed on Jul. 3, 2002.

(30) Foreign Application Priority Data
Dec. 3, 2001 (DK) PA 2001 01789
Dec. 19, 2001 (DK) PA 2001 01917
Dec. 20, 2001 (DK) PA 2001 01925
Jun. 27, 2002 (DK) PA 2002 01006
Jun. 27, 2002 (DK) PA 2002 00999
Jul. 18, 2002 (DK) PA 2002 01117
Feb. 19, 2002 (EP) 02388015.6

(51) Int. Cl. A61K 38/51; A61K 31/00

(52) U.S. Cl. 424/94.5; 514/2; 514/1

(57) ABSTRACT

The invention relates to the use of a combination of a glucokinase activator and a glucagon antagonist for the management, treatment, control, or adjunct treatment of diseases, where increasing glucokinase activity and inhibiting the activity of glucagon is beneficial, such as for management, treatment, control, or adjunct treatment of type 1 diabetes or type 2 diabetes.

### FIELD OF THE INVENTION

This invention relates to the use of a combination of a glucokinase activator and a glucagon antagonist for the management, treatment, control, or adjunct treatment of diseases, where increasing glucokinase activity and inhibiting the activity of glucagon is beneficial, such as for management, treatment, control, or adjunct treatment of type 1 diabetes or type 2 diabetes.

### BACKGROUND OF THE INVENTION

Diabetes is characterised by an impaired glucose metabolism manifesting itself among other things by an elevated blood glucose level in the diabetic patients. Underlying defects lead to a classification of diabetes into two major groups: Type 1 diabetes, or insulin demanding diabetes mellitus (IDDM), which arises when patients lack, β-cells producing insulin in their pancreatic glands, and type 2 diabetes, or non-insulin dependent diabetes mellitus (NIDDM), which occurs in patients with an impaired , β-cell function besides a range of other abnormalities.

Type 1 diabetic patients are currently treated with insulin, while the majority of type 2 diabetic patients are treated either with sulphonylureas that stimulate , β-cell function or with agents that enhance the tissue sensitivity of the patients towards insulin or with insulin. Among the agents applied to enhance tissue sensitivity towards insulin, metformin is a representative example.

Even though sulphonylureas are widely used in the treatment of NIDDM this therapy is, in most instances, not satisfactory: In a large number of NIDDM patients sulphonylureas do not suffice to normalise blood sugar levels and the patients are, therefore, at high risk for acquiring diabetic complications. Also, many patients gradually lose the ability to respond to treatment with sulphonylureas and are thus gradually forced into insulin treatment. This shift of patients from oral hypoglycaemic agents to insulin therapy is usually ascribed to exhaustion of the , β-cells in NIDDM patients.

In normal subjects as well as in diabetic subjects, the liver produces glucose in order to avoid hypoglycaemia. This glucose production is derived either from the release of glucose from glycogen stores or from gluconeogenesis, which is a de novo intracellular synthesis of glucose. In type 2 diabetes, however, the regulation of hepatic glucose output is poorly controlled and is increased, and may be doubled after an overnight fast. Moreover, in these patients there exists a strong correlation between the increased fasting plasma glucose levels and the rate of hepatic glucose production. Similarly, hepatic glucose production will be increased in Type 1 diabetes, if the disease is not properly controlled by insulin treatment.

Since existing forms of therapy of diabetes does not lead to sufficient glycaemic control and therefore are unsatisfactory, there is a great demand for novel therapeutic approaches.

Atherosclerosis, a disease of the arteries, is recognized to be the leading cause of death in the United States and Western Europe. The pathological sequence leading to atherosclerosis and occlusive heart disease is well known. The earliest stage in this sequence is the formation of “fatty streaks” in the carotid, coronary and cerebral arteries and in the aorta. These lesions are yellow in colour due to the presence of lipid deposits found principally within smooth-muscle cells and in macrophages of the intima layer of the arteries and aorta. Further, it is postulated that most of the cholesterol found within the fatty streaks, in turn, give rise to development of the “fibrous plaque”, which consists of accumulated intimal smooth muscle cells laden with lipid and surrounded by extra-cellular lipid, collagen, elastin and proteoglycans. The cells plus matrix form a fibrous cap that covers a deeper deposit of cell debris and more extracellular lipid. The lipid is primarily free and esterified cholesterol. The fibrous plaque forms slowly, and is likely in time to become calcified and necrotic, advancing to the “complicated lesion” which accounts for the arterial occlusion and tendency toward mural thrombosis and arterial muscle spasm that characterize advanced atherosclerosis.

Epidemiological evidence has firmly established hyperlipidemia as a primary risk factor in causing cardiovascular disease (CVD) due to atherosclerosis. In recent years, leaders of the medical profession have placed renewed emphasis on lowering plasma cholesterol levels, and low density lipoprotein cholesterol in particular, as an essential step in prevention of CVD. The upper limits of “normal” are now known to be significantly lower than heretofore appreciated. As a result, large segments of Western populations are now realized to be at particular high risk. Independent risk factors include glucose intolerance, left ventricular hypertrophy, hypertension, and being of the male sex. Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance.

Hypertension (or high blood pressure) is a condition, which occurs in the human population as a secondary symptom to various other disorders such as renal artery stenosis, pheochromocytoma, or endocrine disorders. However, hypertension is also evidenced in many patients in whom the causative agent or disorder is unknown. While such “essential” hypertension is often associated with disorders such as obesity, diabetes, and hypertriglyceridemia, the relationship between these disorders has not been elucidated. Additionally, many patients display the symptoms of high blood pressure in the complete absence of any other signs of disease or disorder.

It is known that hypertension can directly lead to heart failure, renal failure, and stroke (brain haemorrhaging). These conditions are capable of causing short-term death in a patient. Hypertension can also contribute to the development of atherosclerosis and coronary disease. These conditions gradually weaken a patient and can lead to long-term death.

The exact cause of essential hypertension is unknown, though a number of factors are believed to contribute to the onset of the disease. Among such factors are stress, uncontrolled emotions, unregulated hormone release (the renin, angiotensin aldosterone system), excessive salt and water due to kidney malfunction, wall thickening and hypertrophy of the vasculature resulting in constricted blood vessels and genetic factors.

The treatment of essential hypertension has been undertaken bearing the foregoing factors in mind. Thus a broad range of beta-blockers, vasoconstrictors, angiotensin converting enzyme inhibitors and the like have been developed and marketed as antihypertensives. The treatment of hypertension utilizing these compounds has proven beneficial in the prevention of short-interval deaths such as heart failure, renal failure, and brain haemorrhaging. However, the development of atherosclerosis or heart disease due to hypertension over a long period of time remains a problem. This implies that although high blood pressure is being reduced, the underlying cause of essential hypertension is not responding to this treatment.

Hypertension has been associated with elevated blood insulin levels, a condition known as hyperinsulinemia. Insulin, a peptide hormone whose primary actions are to promote glucose utilization, protein synthesis and the formation and storage of neutral lipids, also acts to promote vascular cell growth and increase renal sodium retention, among other things. These latter functions can be accomplished without affecting glucose levels and are known causes of hypertension. Peripheral vasculature growth, for example, can cause constriction of peripheral capillaries, while sodium retention increases blood volume. Thus, the lowering of insulin levels in hyperinsulinemics can prevent abnormal vascular growth and renal sodium retention caused by high insulin levels and thereby alleviates hypertension.

Cardiac hypertrophy is a significant risk factor in the development of sudden death, myocardial infarction, and congestive heart failure. Theses cardiac events are due, at least in part, to increased susceptibility to myocardial injury after ischemia and reperfusion, which can occur in out-patient as well as perioperative settings. There is an unmet medical need to prevent or minimize adverse myocardial perioperative outcomes, particularly perioperative myocardial infarction. Both non-cardiac and cardiac surgery are associated with substantial risks for myocardial infarction or death. Some 7 million patients undergoing non-cardiac surgery are considered to be at risk, with incidences of perioperative death and serious cardiac complications as high as 20-25% in some series. In addition, of the 400,000 patients undergoing coronary by-pass surgery annually, perioperative myocardial infarction is estimated to occur in 5% and death in 1-2%. There is currently no drug therapy in this area, which reduces damage to cardiac tissue from perioperative myocardial ischemia or enhances cardiac resistance to ischemic episodes. Such a therapy is anticipated to be life-saving and reduce hospitalizations, enhance quality of life and to reduce overall health care costs of high risk patients.

Another field for the present invention is obesity or appetite regulation.

Obesity is a well-known risk factor for the development of many very common diseases such as atherosclerosis, hypertension, and diabetes. The incidence of obese people and thereby also these diseases is increasing throughout the entire industrialised world. Except for exercise, diet and food restriction no convincing pharmacological treatment for reducing body weight effectively and acceptably currently exist. However, due to its indirect but important effect as a risk factor in mortal and common diseases it will be important to find treatment for obesity and/or means of appetite regulation.

The term obesity implies an excess of adipose tissue. In this context obesity is best viewed as any degree of excess adiposity that imparts a health risk. The cut off between normal and obese individuals can only be approximated, but the health risk imparted by the obesity is probably a continuum With increasing adiposity. The Framingham study demonstrated that a 20% excess over desirable weight clearly imparted a health risk (Mann GV N.Engl.J.Med 291:226, 1974). In the United States a National Institutes of Health consensus panel on obesity agreed that a 20% increase in relative weight or a body mass index (BMI=body weight in kilograms divided by the square of the height in meters) above the 85th percentile for young adults constitutes a health risk. By the use of these criteria 20 to 30 percent of adult men and 30 to 40 percent of adult women in the United States are obese. (NIH, Ann Intern Med 103:147, 1985).

Even mild obesity increases the risk for premature death, diabetes, hypertension, atherosclerosis, gallbladder disease, and certain types of cancer. In the industrialised western world the prevalence of obesity has increased significantly in the past few decades. Because of the high prevalence of obesity and its health consequences, its prevention and treatment should be a high public health priority.

When energy intake exceeds expenditure, the excess calories are stored in adipose tissue, and if this net positive balance is prolonged, obesity results, i.e. there are two components to weight balance, and an abnormality on either side (intake or expenditure) can lead to obesity.

The regulation of eating behaviour is incompletely understood. To some extent appetite is controlled by discrete areas in the hypothalamus: a feeding centre in the ventrolateral nucleus of the hypothalamus (VLH) and a satiety centre in the ventromedial hypothalamus (VMH). The cerebral cortex receives positive signals from the feeding centre that stimulate eating, and the satiety centre modulates this process by sending inhibitory impulses to the feeding centre. Several regulatory processes may influence these hypothalamic centres. The satiety centre may be activated by the increases in plasma glucose and/or insulin that follow a meal. Meal-induced gastric distension is another possible inhibitory factor. Additionally the hypothalamic centres are sensitive to catecholamines, and beta-adrenergic stimulation inhibits eating behaviour. Ultimately, the cerebral cortex controls eating behaviour, and impulses from the feeding centre to the cerebral cortex are only one input. Psychological, social, and genetic factors also influence food intake.

At present a variety of techniques are available to effect initial weight loss. Unfortunately, initial weight loss is not an optimal therapeutic goal. Rather, the problem is that most obese patients eventually regain their weight. An effective means to establish and/or sustain weight loss is the major challenge in the treatment of obesity today.

Glucokinase (GK) plays an essential role in blood glucose homeostasis. GK catalyses glucose phosphorylation, and is the rate-limiting reaction for glycolysis in hepatocytes and pancreatic β-cells. In liver GK determine the rates of both glucose uptake and glycogen synthesis, and it is also thought to be essential for the regulation of various glucose-responsive genes (Girard, J.et al., Annu Rev Nutr 17, 325-352 (1997)). In the β-cells, GK determines glucose utilization and thus is necessary for glucose-stimulated insulin secretion. GK is also expressed in a population of neurones in the hypothalamus where it might be involved in feeding behaviour and in the gut where it might contribute to the secretion of enteroincretins.

GK has two main distinctive characteristics: its expression, which is limited to tissues that require glucose-sensing (mainly liver and pancreatic , β-cells), and its S_{0 5} for glucose, which is much higher (8-12 mM) than that of the other members of the hexokinase family. Due to these kinetic characteristics, changes in serum glucose levels are paralleled by changes in glucose metabolism in liver which in turn regulate the balance between hepatic glucose output and glucose consumption.

Activators of glucokinase may thus be useful for treating diseases where increasing the activity of glucokinase is beneficial. Thus, there is a need for agents which activate glucokinase and increase glucokinase enzymatic activity. Such agents would be useful for the treatment of diseases, where increasing glucokinase activity is beneficial, such as type I diabetes and type II diabetes.

Activators of glucokinase may also play a role in sensing low glucose levels and generating neurohumoral responses to hypoglycemia and may thus be useful for treating those patients with type 1 diabetes, which has a higher tendency to suffer from hypoglycemia.

WO 00/158293, WO 01/44216, WO 01/83465, WO 01/83478, WO 01/85706, WO 01/85707 and WO 02/08209, to Hoffman-La Roche, disclose compounds as glucokinase activators.

Glucagon is a key hormonal agent that, in co-operation with insulin, mediates homeostatic regulation of the amount of glucose in the blood. Glucagon primarily acts by stimulating certain cells (mostly liver cells) to release glucose when blood glucose levels fall. The action of glucagon is opposite to that of insulin, which stimulates cells to take up and store glucose whenever blood glucose levels rise. Both glucagon and insulin are peptide hormones.

Glucagon is produced in the alpha islet cells of the pancreas and insulin in the beta islet cells. Diabetes mellitus is a common disorder of glucose metabolism. The disease is characterized by hyperglycemia and may be classified as type 1 diabetes, the insulin-dependent form, or type 2 diabetes, which is non-insulin-dependent in character. Subjects with type 1 diabetes are hyperglycemic and hypoinsulinemic, and the conventional treatment for this form of the disease is to provide insulin. However, in some patients with type 1 or type 2 diabetes, absolute or relative elevated glucagon levels have been shown to contribute to the hyperglycemic state. Both in healthy control animals as well as in animal models of type 1 and type 2 diabetes, removal of circulating glucagon with selective and specific antibodies has resulted in reduction of the glycemic level. These studies suggest that glucagon suppression or an action that antagonizes glucagon could be a useful adjunct to conventional treatment of hyperglycemia in diabetic patients. The action of glucagon can be suppressed by providing an antagonist or an inverse agonist, ie substances that inhibit or prevent glucagon-induced responses. The antagonist can be peptidic or non-peptidic in nature.

Native glucagon is a 29 amino acid peptide having the sequence: His-Ser-Glu-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH.

Glucagon exerts its action by binding to and activating its receptor, which is part of the Glucagon-Secretin branch of the 7-transmembrane G-protein coupled receptor family. The receptor functions by activating the adenylyl cyclase second messenger system and the result is an increase in cAMP levels.

Several publications disclose peptides that are stated to act as glucagon antagonists. Probably, the most thoroughly characterized antagonist is DesHis¹[Glu⁹]-glucagon amide (Unson et al., Peptides 10, 1171 (1989); Post et al., Proc. Natl. Acad. Sci. USA 90, 1662 (1993)). Other antagonists are DesHis¹,Phe⁶[Glu⁹]-glucagon amide (Azizh et al., Bioorganic & Medicinal Chem. Lett. 16, 1849 (1995)) and NLeu⁹,Ala^{11,16}-glucagon amide (Unson et al., J. Biol. Chem. 269 (17), 12548 (1994)).

Peptide antagonists of peptide hormones are often quite potent. However, they are generally known not to be orally available because of degradation by physiological enzymes, and poor distribution in vivo. Therefore, orally available non-peptide antagonists of peptide hormones are generally preferred. Among the non-peptide glucagon antagonists, a quinoxaline derivative, (2-styryl-3-[3-(dimethylamino)propylmethylamino]-6,7-dichloroquinoxaline was found to displace glucagon from the rat liver receptor (Collins, J. L. et al., Bioorganic and Medicinal Chemistry Letters 2(9):915-918 (1992)). WO 94/14426 (The Wellcome Foundation Limited) discloses use of skyrin, a natural product comprising a pair of linked 9,10-anthracenedione groups, and its synthetic analogues, as glucagon antagonists. U.S. Pat. No. 4,359,474 (Sandoz) discloses the glucagon inhibiting properties of 1-phenyl pyrazole derivatives. U.S. Pat. No. 4,374,130 (Sandoz) discloses substituted disilacyclohexanes as glucagon inhibiting agents. WO 98/04528 (Bayer Corporation) discloses substituted pyridines and biphenyls as glucagon antagonists. U.S. Pat. No. 5,776,954 (Merck & Co., Inc.) discloses substituted pyridyl pyrroles as glucagon antagonists and WO 98/21957, WO 98/22108, WO 98/22109 and U.S. Pat. No. 5,880,139 (Merck & Co., Inc.) disclose 2,4-diaryl-5-pyridylimidazoles as glucagon antagonists. Furthermore, WO 97/16442 and U.S. Pat. No. 5,837,719 (Merck & Co., Inc.) disclose 2,5-substituted aryl pyrroles as glucagon antagonists. WO 98/24780, WO 98/24782, WO 99/24404 and WO 99/32448 (Amgen Inc.) disclose substituted pyrimidinone and pyridone compounds and substituted pyrimidine compounds, respectively, which are stated to possess glucagon antagonistic activity. Madsen et al. (J. Med. Chem. 1998 (41) 5151-7) discloses a series of 2-(benzimidazol-2-ylthio)-1-(3,4-dihydroxyphenyl)-1-ethanones as competitive human glucagon receptor antagonists. WO 99/01423 and WO 00/39088 (Novo Nordisk A/S) disclose different series of alkylidene hydrazides as glucagon antagonists/-inverse agonists. WO 00/69810, WO 02/00612, WO 02/40444, WO 02/40445 and WO 02/40446 (Novo Nordisk A/S) disclose further classes of glucagon antagonists.

### SUMMARY OF THE INVENTION

The present invention related to the use of a glucokinase activator in combination with a glucagon antagonist for the management, treatment, control and adjunct treatment of diseases where increasing the activity of glucokinase and inhibiting the activity of glucagon is beneficial. Such diseases include type 1 diabetes and type 2 diabetes and diseases and conditions such as hyperglycemia, IGT (impaired glucose tolerance), insulin resistance syndrome, syndrome X, dyslipidemia, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia) hyperlipoproteinemias, and obesity.

The present invention provides pharmaceutical compositions comprising a glucokinase activator and a glucagon antagonist, the combined use of a glucokinase activator and a glucagon antagonist for increasing the activity of glucokinase and inhibiting the activity of glucagon, the combined use of a glucokinase activator and a glucagon antagonist in preparation of a medicament for treating said diseases and conditions and the combined use of a glucokinase activator and a glucagon antagonist in the treatment of said diseases and conditions as well as methods for treating said diseases and conditions, which methods comprise administering to a subject in need thereof an effective amount of a glucokinase activator and a glucagon antagonist. Other embodiments and aspects are as defined below and by the appended claims.

### DEFINITIONS

The term “pharmacologically effective amount” or shall mean that amount of a drug or pharmaceutical agent that will elicit the biological or medical response of a tissue, animal or human that is being sought by a researcher or clinician. This amount can be a therapeutically effective amount. The term “therapeutically effective amount” shall mean that amount of a drug or pharmaceutical agent that will elicit the therapeutic response of an animal or human that is being sought.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compounds to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being.

### DESCRIPTION OF THE INVENTION

The present invention related to the use of a glucokinase activator in combination with a glucagon antagonist for the management, treatment, control and adjunct treatment of diseases where increasing the activity of glucokinase and inhibiting the activity of glucagon is beneficial. Such diseases include type 1 diabetes and type 2 diabetes and diseases and conditions such as hyperglycemia, IGT (impaired glucose tolerance), insulin resistance syndrome, syndrome X, dyslipidemia, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia) hyperlipoproteinemias, and obesity. According to the invention, a glucokinase activator in combination with a glucagon antagonist may also be used for the delaying or prevention of the progression from IGT to type 2 diabetes and for the delaying or prevention of the progression from non-insulin requiring type 2 diabetes to insulin requiring type 2 diabetes.

The present invention also relates to use of a combination of a glucokinase activator and a glucagon antagonist in preparation of a medicament for treating said diseases and conditions.

The present invention also relates to use of a glucokinase activator in preparation of a medicament to be used in combination with a glucagon antagonist in the treatment of said diseases and conditions.

The present invention also relates to use of a glucagon antagonist in preparation of a medicament to be used in combination with a glucokinase activator in the treatment of said diseases and conditions.

The present invention also relates to methods for treating said diseases and conditions, which methods comprise administering to a subject in need thereof a therapeutically effective amount of a glucokinase activator and a effective amount of a glucagon antagonist.

The present invention also relates to a method for activating glucokinase and inhibiting the activity of glucagon in a patient in need thereof, which method comprises administering to a subject in need thereof a pharmacologically effective amount of a glucokinase activator and a pharmacologically effective amount of a glucagon antagonist. The phrase “a subject in need thereof” includes mammalian subjects, preferably humans, for whom an activation of glucokinase and inhibition of the activity of glucagon is beneficial.

The present invention also relates to a method for lowering blood glucose in a patient in need thereof, which method comprises administering to a subject in need thereof a pharmacologically effective amount of a glucokinase activator and a pharmacologically effective amount of a glucagon antagonist. The phrase “a subject in need thereof” includes mammalian subjects, preferably humans, who either suffer from elevated blood glucose or are at risk of suffering from elevated blood glucose.

The present invention also relates to a method for treating diseases where increasing the activity of glucokinase and inhibiting the activity of glucagon is beneficial. Such diseases include type 1 diabetes and type 2 diabetes and diseases and conditions such as hyperglycemia, IGT (impaired glucose tolerance), insulin resistance syndrome, syndrome X, dyslipidemia, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia) hyperlipoproteinemias, and obesity.

As used herein, the phrase “a subject in need thereof” includes mammalian subjects, preferably humans, who either suffer from one or more of the aforesaid diseases or disease states or are at risk for such. Accordingly, in the context of the therapeutic method of the invention, this method also is comprised of a method for treating a mammalian subject prophylactically, or prior to the onset of diagnosis such disease(s) or disease state(s).

In one embodiment, the pharmacologically effective amount is a therapeutically effective amount. Other embodiments of such methods will be clear from the following description.

Glucokinase activators may be identified by use of Glucokinase Activity Assay (I) disclosed herein. A selection of compounds, such as for instance a library of compounds, may be screened according to Glucokinase Activity Assay (I), for instance in a high-throughput-screening, and glucokinase activators may thereby be identified. glucokinase activators according to the present invention may, at a concentration of at or below 30 μM give 1.5-fold higher glucokinase activity than the result from the assay without compound, such as 2.0-fold higher, for example 2.5-fold higher, such as 3.0 fold higher, for example 5.0 fold higher.

Glucagon antagonist may be identified by use of Glucagon Binding Assay (I) or Glucagon Binding Assay (II) disclosed herein. A selection of compounds, such as for instance a library of compounds, may be screened according to assay Glucagon Binding Assay (I) or Glucagon Binding Assay (II), for instance in a high-throughput-screening, and glucagon antagonists may thereby be identified. Glucagon antagonists according to the present invention may have an IC₅₀ value of no greater than 5 μM, such as less than 1 μM, for example less than 500 nM, such as less than 100 nM as determined by the Glucagon Binding Assay (I) or Glucagon Binding Assay (II) disclosed herein.

In one embodiment, the glucokinase activator is a glucokinase activator as described in WO 00/58293, WO 01/44216, WO 01/83465, WO 01/83478, WO 01/85706, or WO 01/85707, to Hoffman-La Roche.

In one embodiment, the glucagon antagonist is 2-styryl-3-[3-(dimethylamino)-propylmethylamino]-6,7-dichloroquinoxaline.

In one embodiment, the glucagon antagonist is a glucagon antagonist as described in WO 94/14426 (The Wellcome Foundation Limited).

In one embodiment, the glucagon antagonist is a glucagon antagonist as described in U.S. Pat. No. 4,359,474 or U.S. Pat. No. 4,374,130 (Sandoz).

In one embodiment, the glucagon antagonist is a glucagon antagonist as described in WO 98/04528 (Bayer Corporation).

In one embodiment, the glucagon antagonist is a glucagon antagonist as described in U.S. Pat. No. 5,776,954, WO 98/21957, WO 98/22108, WO 98/22109, WO 97/16442, U.S. Pat. No. 5,837,719 or U.S. Pat. No. 5,880,139 (Merck & Co., Inc.).

In one embodiment, the glucagon antagonist is a glucagon antagonist as described in WO 98/24780, WO 98/24782, WO 99/24404 and WO 99/32448 (Amgen Inc.).

In one embodiment, the glucagon antagonist is a glucagon antagonist as described in Madsen et al. (J. Med. Chem. 1998 (41) 5151-7).

In one embodiment, the glucagon antagonist is a glucagon antagonist as described in WO 99/01423 and WO 00/39088, WO 00/69810, WO 02/00612, WO 02/40444, WO 02/40445 or WO 02/40446 (Novo Nordisk A/S).

The glucokinase activator and the glucagon antagonist for use according to the present invention may be administered as separate pharmaceutical compositions or as parts of the same pharmaceutical composition.

The treatment may also be a combination of administration of a pharmaceutical composition comprising the glucokinase activator (but comprising no glucagon antagonist), and a pharmaceutical composition comprising the glucokinase activator and the glucagon antagonist. The treatment may also be a combination of administration of a pharmaceutical composition comprising the glucagon antagonist (but comprising no glucagon antagonist), and a pharmaceutical composition comprising the glucokinase activator and the glucagon antagonist. The treatment may also be a combination of administration of a pharmaceutical composition comprising the glucokinase activator (but comprising no glucagon antagonist), a pharmaceutical composition comprising the glucagon antagonist (but comprising no glucagon antagonist), and a pharmaceutical composition comprising the glucokinase activator and the glucagon antagonist.

According to the selected regime, the glucokianse activator and the glucagon antagonist may be administered at the same time or at different times (which may coincide one or more times). The dosing regimen of the glucokinase activator and the glucagon antagonist will depend on the mode of administration, on the therapy desired, the form in which the glucokinase activator and the glucagon antagonist are administered, the subject to be treated and the body weight of the subject to be treated, and the preference and experience of the physician or veterinarian in charge.

In one aspect of the invention the glucokinase activator and the glucagon antagonist are administered in combination with one or more further active substances in any suitable ratios. Such further active agents may be selected from antidiabetic agents, antihyper-lipidemic agents, antiobesity agents, antihypertensive agents and agents for the treatment of complications resulting from or associated with diabetes.

Suitable antidiabetic agents include insulin, GLP-1 (glucagon like peptide-1) derivatives such as those disclosed in WO 98/08871 (Novo Nordisk A/S), which is incorporated herein by reference, as well as orally active hypoglycemic agents.

Suitable orally active hypoglycemic agents include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic β-cells eg potassium channel openers such as those disclosed in WO 97/26265, WO 99/03861 and WO 00/37474 (Novo Nordisk A/S) which are incorporated herein by reference, potassium channel openers, such as ormitiglinide, potassium channel blockers such as nateglinide or BTS-67582, all of which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists such as ALRT-268, LG-1268 or LG-1069.

In one embodiment of the invention, the glucokinase activator and the glucagon antagonist are administered in combination with insulin or insulin analogues.

In one embodiment of the invention, the glucokinase activator and the glucagon antagonist are administered in combination with a sulphonylurea eg tolbutamide, chlorpropamide, tolazamide, glibenclamide, glipizide, glimepiride, glicazide or glyburide.

In one embodiment of the invention, the glucokinase activator and the glucagon antagonist are administered in combination with a biguanide eg metformin.

In one embodiment of the invention, the glucokinase activator and the glucagon antagonist are administered in combination with a meglitinide eg repaglinide or senaglinide/nateglinide.

In one embodiment of the invention, the glucokinase activator and the glucagon antagonist are administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097 (DRF-2344), WO 97/41119, WO 97/41120, WO 00/41121 and WO 98/45292 (Dr. Reddy's Research Foundation), which are incorporated herein by reference.

In one embodiment of the invention the glucokinase activator and the glucagon antagonist may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313 (NN622/DRF-2725), WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In one embodiment of the invention the glucokinase activator and the glucagon antagonist are administered in combination with an a-glucosidase inhibitor eg voglibose, emiglitate, miglitol or acarbose.

In one embodiment of the invention the glucokinase activator and the glucagon antagonist are administered in combination with a glycogen phosphorylase inhibitor eg the compounds described in WO 97/09040 (Novo Nordisk A/S).

In one embodiment of the invention the glucokinase activator and the glucagon antagonist are administered in combination with an agent acting on the ATP-dependent potassium channel of the pancreatic β-cells eg tolbutamide, glibenclamide, glipizide, glicazide, BTS-67582 or repaglinide.

In one embodiment of the invention the glucokinase activator and the glucagon antagonist are administered in combination with nateglinide.

In one embodiment of the invention the glucokinase activator and the glucagon antagonist are administered in combination with an antihyperlipidemic agent or a antilipidemic agent eg cholestyramine, colestipol, clofibrate, gemfibrozil, lovastatin, pravastatin, simvastatin, probucol or dextrothyroxine.

In another aspect of the invention, the glucokinase activator and the glucagon antagonist are administered in combination with more than one of the above-mentioned compounds eg in combination with metformin and a sulphonylurea such as glyburide; a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

Furthermore, the glucokinase activator and the glucagon antagonist may be administered in combination with one or more antiobesity agents or appetite regulating agents.

Such agents may be selected from the group consisting of CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, which are incorporated herein by reference, exendin-4, GLP-1 agonists and ciliary neurotrophic factor. Further antiobesity agents are bupropion (antidepressant), topiramate (anticonvulsant), ecopipam (dopamine D1/D5 antagonist) and naltrexone (opioid antagonist).

In one embodiment of the invention the antiobesity agent is leptin.

In one embodiment of the invention the antiobesity agent is a serotonin and norepinephrine reuptake inhibitor eg sibutramine.

In one embodiment of the invention the antiobesity agent is a lipase inhibitor eg orlistat.

In one embodiment of the invention the antiobesity agent is an adrenergic CNS stimulating agent eg dexamphetamine, amphetamine, phentermine, mazindol phendimetrazine, diethylpropion, fenfluramine or dexfenfluramine.

Furthermore, the glucokinase activator and the glucagon antagonist may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19th Edition, Gennaro, Ed., Mack Publishing Co., Easton, PA, 1995.

These additional compounds may be administered as separate pharmaceutical compositions or as part of the pharmaceutical composition comprising the glucokinase activator or as part of the pharmaceutical composition comprising the glucagon antagonist or as part of the pharmaceutical composition comprising the combination of the glucokinase activator and the glucagon antagonist.

It should be understood that any suitable combination of the glucokinase activator and the glucagon antagonist with diet and/or exercise, one or more of the above-mentioned compounds and optionally one or more other active substances, such as for instance one or more additional glucokinase activators and/or glucagon antagonists are considered to be within the scope of the present invention.

The use of a combination of a glucokinase activator and a glucagon antagonist may give synergistic results, that is, more than additive results, in the treatment of said diseases compared to the treatment with either agent alone.

Pharmaceutical Compositions

In one aspect, the present invention includes within its scope pharmaceutical compositions comprising a glucokinase activator and a glucagon antagonist, or pharmaceutically acceptable salts thereof, as active ingredients, together with a pharmaceutically acceptable carrier or diluent.

Optionally, the pharmaceutical composition of the invention may comprise a glucokinase activator and a glucagon antagonist combined with one or more other compounds.

Pharmaceutical compositions containing a glucokinase activator and a glucagon antagonist may be prepared by conventional techniques, e.g. as described in Remington: The Science and Practise of Pharmacy, 19^{th} Ed., 1995. The compositions may appear in conventional forms, for example capsules, tablets, aerosols, solutions, suspensions or topical applications.

Typical compositions include a glucokinase activator and a glucagon antagonist, or pharmaceutically acceptable salts thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compounds will usually be mixed with a carrier, or diluted by a carrier, or enclosed within a carrier which may be in the form of a ampoule, capsule, sachet, paper, or other container. When the carrier serves as a diluent, it may be solid, semi-solid, or liquid material which acts as a vehicle, excipient, or medium for the active compounds. The active compounds can be adsorbed on a granular solid container for example in a sachet. Some examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatine, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compounds to the appropriate or desired site of action, such as oral, nasal, pulmonary, buccal, subdermal, intradermal, transdermal or parenteral e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatine capsule in powder or pellet form or it can be in the form of a troche or lozenge. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a glucokinase activator and a glucagon antagonist dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compounds dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet which may be prepared by conventional tabletting techniques may contain:

Core:

| | | |
|---|---|---|
| Active compounds (as free compounds or salts thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

The glucokinase activator and the glucagon antagonist, may be administered according to the present invention to a mammal, especially a human in need of such treatment, prevention, elimination, alleviation or amelioration of obesity. Such mammals include also animals, both domestic animals, e.g. household pets, and non-domestic animals such as wildlife.

The glucokinase activator and the glucagon antagonist may be effective over a wide dosage range.

For example, in the treatment of adult humans, dosages from about 0.01 mg to about 1000 mg, such as from about 0.05 mg to about 1000 mg, for example from about 0.05 mg to about 500 mg, such as from about 0.1 mg to about 500 mg, for example from about 0.1 mg to about 250 mg, such as from about 0.5 mg to about 250 mg, for example from about 0.5 mg to 100 mg, such as from about 1 mg to 100 mg, for example from about 1 mg to 50 mg, such as from about 1 mg to 25 mg of each active compound pr kg body weight per day may be used. The exact dosage of respectively the glucokinase activator and the glucagon antagonist will depend upon the frequency and mode of administration, on the therapy desired, the form in which the glucokinase activator and the glucagon antagonist are administered, the potency of the glucokinase activator and the glucagon antagonist, the sex, age, weight and general condition of the subject to be treated, the nature and severity of the condition treated and any concomitant diseases to be treated and other factors evident to those skilled in the art, and the preference and experience of the physician or veterinarian in charge.

A typical oral dosage may also be in the range of from about 0.001 mg to about 100 mg, for example from about 0.01 mg to about 50 mg, such as from about 0.05 mg to about 10 mg per kg body weight per day administered in one or more dosages such as 1 to 3 dosages. In choosing a regimen for patients it may frequently be necessary to begin with a higher dosage and when the condition is under control to reduce the dosage.

The formulations may conveniently be presented in unit dosage form by methods known to those skilled in the art. A typical unit dosage form for oral administration one or more times per day such as 1 to 3 times per day may contain from about 0.01 mg to about 1000 mg, such as from about 0.05 mg to about 1000 mg, for example from about 0.05 mg to about 500 mg, such as from about 0.1 mg to about 500 mg, for example from about 0.1 mg to about 250 mg, such as from about 0.5 mg to about 250 mg, for example from about 0.5 mg to 100 mg, such as from about 1 mg to 100 mg, for example from about 1 mg to 50 mg, such as from about 1 mg to 25 mg of each active compound either in separate formulations or as a combined formulation as described above.

Generally, the glucokinase activator and the glucagon antagonist are dispensed in separate unit dosage forms comprising from about 0.01 mg to about 1000 mg, such as from about 0.05 mg to about 1000 mg, for example from about 0.05 mg to about 500 mg, such as from about 0.1 mg to about 500 mg, for example from about 0.1 mg to about 250 mg, such as from about 0.5 mg to about 250 mg, for example from about 0.5 mg to 100 mg, such as from about 1 mg to 100 mg, for example from about 1 mg to 50 mg, such as from about 1 mg to 25 mg of each active ingredient together with a pharmaceutically acceptable carrier or diluent per unit dosage or together in a combined unit dosage form comprising from about from about 0.01 mg to about 1000 mg, such as from about 0.05 mg to about 1000 mg, for example from about 0.05 mg to about 500 mg, such as from about 0.1 mg to about 500 mg, for example from about 0.1 mg to about 250 mg, such as from about 0.5 mg to about 250 mg, for example from about 0.5 mg to 100 mg, such as from about 1 mg to 100 mg, for example from about 1 mg to 50 mg, such as from about 1 mg to 25 mg, of each active ingredient together with a pharmaceutically acceptable carrier per unit dosage.

Any novel feature or combination of features described herein is contemplated within the scope of this invention.

### EXAMPLES

Glucokinase Activity Assay (I)

Glucokinase activity is assayed spectrometrically coupled to glucose 6-phosphate dehydrogenase to determine compound activation of glucokinase. The final assay contains 50 mM Hepes, pH 7.1, 50 mM KCl, 5 mM MgCl₂, 2 mM dithiothreitol, 0.6 mM NADP, 1 mM ATP, 0.195 μM G-6-P dehydrogenase (from Roche, 127 671), 15 nM recombinant human glucokinase. The glucokinase is human liver glucokinase N-terminally truncated with an N-terminal His-tag ((His)₈-VEQILA . . . Q466) and is expressed in E.coli as a soluble protein with enzymatic activity comparable to liver extracted GK.

The purification of His-tagged human glucokinase (hGK) was performed as follows: The cell pellet from 50 ml E. coli culture was resuspended in 5 ml extraction buffer A (25 mM HEPES, pH 8.0, 1 mM MgCl₂, 150 mM NaCl, 2 mM mercaptoethanol) with addition of 0.25 mg/ml lysozyme and 50 μg/ml sodium azide. After 5 minutes at room temperature 5 ml of extraction buffer B (1.5 M NaCl, 100 mM CaCl₂, 100 mM MgCl₂, 0.02 mg/ml DNase 1, protease inhibitor tablet (Complete® 1697498): 1 tablet pr. 20 ml buffer) was added. The extract was then centrifuged at 15.000 g for 30 minutes. The resulting supernatant was loaded on a 1 ml Metal Chelate Affinity Chromatography (MCAC) Column charged with Ni²⁺. The column is washed with 2 volumes buffer A containing 20 mM imidazole and the bound his-tagged hGK is subsequently eluted using a 20 minute gradient of 20 to 500 mM imididazol in buffer A. Fractions are examined using SDS-gel-electrophoresis, and fractions containing hGK (MW: 52 KDa) are pooled. Finally a gelfiltration step is used for final polishing and buffer exchange. hGK containing fractions are loaded onto a Superdex 75 (16/60) gelfiltration column and eluted with Buffer B (25 mM HEPES, pH 8.0, 1 mM MgCl₂, 150 mM NaCl, 1 mM Dithiothreitol). The purified hGK is examined by SDS-gel electrophoresis and MALDI mass spectrometry and finally 20% glycerol is added before freezing. The yield from 50 ml E. coli culture is generally approximately 2-3 mg hGK with a purity>90%.

The compound to be tested is added into the well in final 2.5% DMSO concentration in an amount sufficient to give a desired concentration of compound, for instance 50 μM. The reaction starts after glucose is added to a final concentration of 2, 5, 10 or 15 mM. The assay uses a 96-well UV plate and the final assay volume used is 200 μl/well. The plate is incubated at 25° C. for 5 min and kinetics is measured at 340 nm in SpectraMax every 30 seconds for 5 minutes. Results for each compound are expressed as the fold activation of the glucokinase activity compared to the activation of the glucokinase enzyme in an assay without test compound present after having been subtracted from a “blank”, which is without glucokinase enzyme and without compound. A compound, which at a concentration of at or below 30 μM gives 1.5-fold higher glucokinase activity than the result from the assay without compound, is deemed to be an activator of glucokinase.

Glucagon Binding Assay (I)

Receptor binding are assayed using cloned human receptor (Lok et al., Gene 140, 203-209 (1994)). The receptor inserted in the pLJ6′ expression vector using EcoRI/SSt1 restriction sites (Lok et al.) is expressed in a baby hamster kidney cell line (A3 BHK 570-25). Clones are selected in the presence of 0.5 mg/ml G-418 and are shown to be stable for more than 40 passages. The K_{d} is shown to be 0.1 nM.

Plasma membranes are prepared by growing cells to confluence, detaching them from the surface and resuspending the cells in cold buffer (10 mM Tris/HCl, pH 7.4 containing 30 mM NaCl, 1 mM dithiothreitol, 5 mg/l leupeptin (Sigma), 5 mg/l pepstatin (Sigma), 100 mg/I bacitracin (Sigma) and 15 mg/l recombinant aprotinin (Novo Nordisk A/S)), homogenization by two 10-s bursts using a Polytron PT 10-35 homogenizer (Kinematica), and centrifugation upon a layer of 41 w/v % sucrose at 95.000×g for 75 min. The white band located between the two layers is diluted in buffer and centrifuged at 40.000×g for 45 min. The precipitate containing the plasma membranes is suspended in buffer and stored at −80° C. until use.

Glucagon is iodinated according to the chloramine T method (Hunter and Greenwood, Nature 194, 495 (1962)) and purified using anion exchange chromatography (Jørgensen et al., Hormone and Metab. Res. 4, 223-224 (1972). The specific activity is 460 μCi/μg on the day of iodination. Tracer is stored at −18° C. in aliquots and used immediately after thawing.

Binding assays are carried out in triplicate in filter microtiter plates (MADV N65, Millipore). The buffer is 50 mM HEPES, 5 mM EGTA, 5 mM MgCl₂, 0.005% Tween 20, pH 7.4. Glucagon is dissolved in 0.05 M HCl, added an equal amount (w/w) of human serum albumin and freeze-dried. On the day of use, it is dissolved in water and diluted in buffer to the desired concentrations.

Test compounds are dissolved and diluted in DMSO. 140 μl buffer, 25 μl glucagon or buffer, and 10 μl DMSO or test compound are added to each well. Tracer (50.000 cpm) is diluted in buffer and 25 μl is added to each well. 1-4 μg freshly thawed plasma membrane protein diluted in buffer is then added in aliquots of 25 μl to each well. Plates are incubated at 30° C. for 2 hours. Non-specific binding is determined with 10⁻⁶ M of glucagon. Bound tracer and unbound tracer are then separated by vacuum filtration (Millipore vacuum manifold). The plates are washed with 2×100 μl buffer/well. The plates are air dried for a couple of hours, whereupon the filters are separated from the plates using a Millipore Puncher. The filters are counted in a gamma counter.

Functional Glucagon Assay (I)

The functional assay is carried out in 96 well microtiter plates (tissue culture plates, Nunc). The resulting buffer concentrations in the assay are 50 mM Tris/HCl, 1 mM EGTA, 1.5 mM MgSO₄, 1.7 mM ATP, 20 μM GTP, 2 mM IBMX, 0.02% Tween-20 and 0.1% human serum albumin. pH was 7.4. Glucagon and proposed antagonist are added in aliquots of 35 μl diluted in 50 mM Tris/HCl, 1 mM EGTA, 1.85 mM MgSO₄, 0.0222% Tween-20 and 0.111% human serum albumin, pH 7.4. 20 μl of 50 mM Tris/HCl, 1 mM EGTA, 1.5 mM MgSO₄, 11.8 mM ATP, 0.14 mM GTP, 14 mM IBMX and 0.1% human serum albumin, pH 7.4 was added. GTP was dissolved immediately before the assay.

50 μl containing 5 μg of plasma membrane protein was added in a Tris/HCl, EGTA, MgSO₄, human serum albumin buffer (the actual concentrations are dependent upon the concentration of protein in the stored plasma membranes).

The total assay volume is 140 μl. The plates are incubated for 2 hours at 37° C. with continuous shaking. Reaction is terminated by addition of 25 μl 0.5 N HCl. cAMP is measured by the use of a scintillation proximity kit (Amersham).

Glucagon Binding Assay (II)

BHK (baby hamster kidney cell line) cells are transfected with the human glucagon receptor and a membrane preparation of the cells is prepared. Wheat Germ Agglutinin derivatized SPA beads containing a scintillant (WGA beads) (Amersham) bound the membranes. ¹²⁵I-glucagon bound to human glucagon receptor in the membranes and excited the scintillant in the WGA beads to light emission. Glucagon or samples binding to the receptor competed with ¹²⁵I-glucagon.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/I bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The glucagon binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl glucagon or test compound (in DMSO) are added to each well. 50 μl tracer (¹²⁵l-porcine glucagon, 50.000 cpm) and 50 μl membranes (7.5 μg) containing the human glucagon receptor are then added to the wells. Finally 50 μl WGA beads containing 1 mg beads are transferred to the well. The opti plates are incubated for 4 hours on a shaker and then settled for 8-48 hours. The opti plates are counted in a Topcounter. Non-specific binding is determined with 500 nM of glucagon.

While the invention has been described and illustrated with reference to certain embodiments thereof, those skilled in the art will appreciate that various changes, modifications and substitutions can be made therein without departing from the spirit and scope of the invention. For example, effective dosages other than the dosages as set forth herein may be applicable as a consequence of variations in the responsiveness of the mammal being treated for said disease(s) or condition(s). Likewise, the specific pharmacological responses observed may vary according to and depending on the particular active compounds selected or whether there are present pharmaceutical carriers, as well as the type of formulation and mode of administration employed, and such expected variations or differences in the results are contemplated in accordance with the objects and practices of the present invention.

1. Use of a glucokinase activator and a glucagon antagonist for the treatment of a disease or condition, where increasing glucokinase activity and inhibiting the activity of glucagon is beneficial for the subject to be treated.

2. Use of a glucokinase activator in preparation of a medicament for use in combination with a glucagon antagonist for treating a disease or condition, where increasing glucokinase activity and inhibiting the activity of glucagon is beneficial for the subject to be treated.

3. Use of a glucagon antagonist in preparation of a medicament for use in combination with a glucokinase activator for treating a disease or condition, where increasing glucokinase activity and inhibiting the activity of glucagon is beneficial for the subject to be treated.

4. Use of a glucokinase activator and a glucagon antagonist in preparation of a medicament for treating a disease or condition, where increasing glucokinase activity and inhibiting the activity of glucagon is beneficial for the subject to be treated.

5. Use according to any of claims 1 to 4, where the disease or condition is type 1 diabetes and type 2 diabetes and diseases and conditions such as hyperglycemia, IGT (impaired glucose tolerance), insulin resistance syndrome, syndrome X, dyslipidemia, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia) hyperlipoproteinemias, or obesity.

6. Use according to claim 5, where the disease or condition is hyperglycemia.

7. Use according to claim 5, where the disease or condition is IGT.

8. Use according to claim 5, where the disease or condition is dyslipidemia.

9. Use according to claim 5, where the disease or condition is obesity.

10. Use according to claim 5, where the disease or condition is type 1 diabetes.

11. Use according to claim 5, where the disease or condition is type 2 diabetes.

12. Use of a glucokinase activator and a glucagon antagonist for reducing blood glucose in a subject in need thereof.

13. Use of a glucokinase activator in preparation of a medicament for use in combination with a glucagon antagonist for reducing blood glucose in a subject in need thereof.

14. Use of a glucagon antagonist in preparation of a medicament for use in combination with a glucokinase activator for reducing blood glucose in a subject in need thereof.

15. Use of a glucokinase activator and a glucagon antagonist in preparation of a medicament for reducing blood glucose in a subject in need thereof.

16. Use according to any of claims 1 to 15, wherein the glucagon antagonist is a peptide glucagon antagonist.

17. Use according to any of claims 1 to 15, wherein the glucagon antagonist is a non-peptide glucagon antagonist.

18. Use according to any of claims 1 to 17, wherein the glucokinase activator at a concentration of at or below 30 μM gives a 1.5-fold higher glucokinase activity in Glucokinase Activity Assay (I) than is measured in Glucokinase Activity Assay (I) without test compound present.

19. Use according to claim 18, wherein the glucokinase activator at a concentration of at or below 30 μM gives a 2.5-fold higher glucokinase activity in Glucokinase Activity Assay (I) than is measured in Glucokinase Activity Assay (I) without test compound present.

20. Use according to claim 19, wherein the glucokinase activator at a concentration of at or below 30 μM gives a 3.0-fold higher glucokinase activity in Glucokinase Activity Assay (I) than is measured in Glucokinase Activity Assay (I) without test compound present.

21. Use according to claim 20, wherein the glucokinase activator at a concentration of at or below 30 μM gives a 5.0-fold higher glucokinase activity in Glucokinase Activity Assay (I) than is measured in Glucokinase Activity Assay (I) without test compound present.

22. Use according to any of claims 1 to 21, wherein the glucagon antagonist has an IC₅₀ value in Glucagon Binding Assay (I) or Glucagon Binding Assay (II) of no greater than 5 μM.

23. Use according to claim 22, wherein the glucagon antagonist has an IC₅₀ value in Glucagon Binding Assay (I) or Glucagon Binding Assay (II) of less than 1 μM.

24. Use according to claim 23, wherein the glucagon antagonist has an IC₅₀ value in Glucagon Binding Assay (I) or Glucagon Binding Assay (II) of less than 500 nm.

25. Use according to claim 24, wherein the glucagon antagonist has an IC₅₀ value in Glucagon Binding Assay (I) or Glucagon Binding Assay (II) of less than 100 nm.

26. A method for treating diabetes or a disease or a condition related to diabetes, said method comprising administering to a subject in need of such treatment a first amount of a glucokinase activator and a second amount of a glucagon antagonist, wherein said first and second amounts together are effective to treat said diabetes or diabetes-related disease or condition.

27. A method according to claim 26, wherein the diabetes or diabetes-related disease or condition is selected from the group consisting of: type 1 diabetes, type 2 diabetes, hyperglycemia, IGT (impaired glucose tolerance), insulin resistance syndrome, syndrome X, dyslipidemia, dyslipoproteinemia (abnormal lipoproteins in the blood), dyslipidemia, hyperlipidemia, hyperlipoproteinemia (excess of lipoproteins in the blood), and obesity.

28. A method according to claim 27, wherein the disease or condition is hyperglycemia.

29. A method according to claim 27, wherein the disease or condition is IGT.

30. A method according to claim 27, wherein the disease or condition is dyslipidemia.

31. A method according to claim 27, wherein the disease or condition is obesity.

32. A method according to claim 27, wherein the disease or condition is type 1 diabetes.

33. A method according to claim 27, wherein the disease or condition is type 2 diabetes.

34. A method according to claim 26, wherein the glucagon antagonist is a peptide.

35. A method according to claim 26, wherein the glucagon antagonist is not a peptide.

36. A method according to claim 26, wherein the glucokinase activator exhibits a level of activation of glucokinase in a glucokinase activity assay of at least about 1.5 when used in a at a concentration of 30 μM or below.

37. A method according to claim 36, wherein the glucokinase activator exhibits a level of activation of at least about 2.5.

38. A method according to claim 37, wherein the glucokinase activator exhibits a level of activation of at least about 3.0.

39. A method according to claim 38, wherein the glucokinase activator exhibits a level of activation of at least about 5.0.

40. A method according to claim 26, wherein the glucagon antagonist has an IC₅₀ value in a glucagon-binding assay of no greater than about 5 μM.

41. A method according to claim 40, wherein the glucagon antagonist has an IC50 value in a glucagon binding assay of less than about 1 μM.

42. A method according to claim 41, wherein the glucagon antagonist has an IC₅₀ value in a glucagon-binding assay of less than about 500 nm.

43. A method according to claim 42, wherein the glucagon antagonist has an IC₅₀ value in a glucagon-binding assay of less than about 100 nm.
